(19) United States
(12) Patent Application Publication
(10) Pub. No.: US 2026/0272377 A1
(43) Pub. Date: Sep. 17, 2026

(54) IMPLANTABLE MEDICAL DEVICE

(71) Applicant: Siemens Healthineers AG, Forchheim (DE)

(72) Inventors: M. MOHANA KRISHNA, Bangalore (IN); Lokesh RAJAGOPAL, Bangalore (IN); Hemnath VENKATESAN, Bangalore (IN)

(73) Assignee: Siemens Healthineers AG, Forchheim (DE)

(21) Appl. No.: 19/563,162

(22) Filed: Mar. 11, 2026

(30) Foreign Application Priority Data
Mar. 12, 2025 (EP) 25163194.1

(51) Int. Cl. A61B 5/00; A61B 5/02; A61B 5/05; A61F 2/48; A61F 2/86

(52) U.S. Cl. CPC A61B 5/4851; A61B 5/0031; A61B 5/02007; A61B 5/05; A61B 5/6862; A61B 5/6876; A61F 2/482; A61F 2/86; A61F 2230/0069

(57) ABSTRACT

An implantable medical device comprises a structure having a cylindrical shape and an inner lumen, and a number of radio transponders, at least one radio transponder of the number of radio transponders being configured to transmit a radio signal having a certain frequency to a receiver.

### CROSS-REFERENCE TO RELATED APPLICATION(S)

The present application claims priority under 35 U.S.C. § 119 to European Patent Application No. 25163194.1, filed Mar. 12, 2025, the entire contents of which is incorporated herein by reference.

### FIELD

One or more example embodiments relates to an implantable medical device comprising a structure having a cylindrical shape and an inner lumen, e.g. a stent, particularly adapted for restenosis identification. Furthermore, one or more example embodiments relates to a system, particularly adapted for restenosis identification, the system including such an implantable medical device and a number of readers being coupleable with the implantable medical device.

### RELATED ART

After an implantable medical device, e.g. a stent or coronary stent, is placed in an artery, several potential complications may arise after the procedure. These potential complications may include restenosis, i.e., a re-narrowing of the treated artery, and stent thrombosis, i.e., the formation of a blood clot within the stent.

To identify the above issues, an invasive diagnostic angiogram is conventionally used. This may result in high radiation redoing the entire procedure.

In particular, in many cases, a repeat of the complete angiography procedures, including ECG, echocardiography, computed tomography, and/or angiography may be needed to re-assess the situation. In large cases of stent restenosis, the patient needs to undergo an intravascular ultrasound or optical coherence tomography (OCT). Disadvantageously, there is no conventional solution available without a radiation exposure for the patient.

Document U.S. Pat. No. 9,188,558B2 discloses a method and a system of monitoring environmental exposure of stents using radiofrequency identification. The disclosed method of monitoring a stent comprises: obtaining readings of an environmental parameter from a sensor adjacent to a stent, the sensor positioned within or on a container including the stent, wherein an RFID tag located within or on the container receives the readings, and wherein the RFID tag and the sensor are integrated as one unit or the RFID tag and the sensor are separate units, transmitting the readings from the RFID tag to a transceiver, wherein a maximum tolerance of the stent for the environmental parameter is stored on the RFID tag, and comparing the readings to the maximum tolerance for the environmental parameter, wherein the environmental parameter is selected from the group consisting of vibration, and shock.

### SUMMARY

One or more example embodiments provides an improved implantable medical device.

### BRIEF DESCRIPTION OF THE DRAWINGS

Further embodiments, features and advantages of the present invention will become apparent from the subsequent description and dependent claims, taken in conjunction with the accompanying drawings, in which:

FIG. 1 shows a schematic illustration of a first embodiment of an implantable medical device;

FIG. 2 shows a further view of the first embodiment of the implantable medical device according to FIG. 1;

FIG. 3 shows a schematic illustration of a second embodiment of an implantable medical device having a plurality of radio transponders;

FIG. 4 shows a schematic illustration of a third embodiment of an implantable medical device having a plurality of radio transponders being implanted in a vessel;

FIG. 5 shows a further view of the third embodiment of FIG. 4 illustrating stenosis identification using a multi direction detection;

FIG. 6 shows a schematic illustration of a first embodiment of a system adapted for restenosis identification; and

FIG. 7 shows a schematic illustration of a second embodiment of a system adapted for restenosis identification.

In the Figures, like reference numerals designate like or functionally equivalent elements, unless otherwise indicated.

### DETAILED DESCRIPTION

According to one or more example embodiments, an implantable medical device is suggested, wherein the implantable medical device includes a structure having a cylindrical shape and an inner lumen, and a number N1 of radio transponders, with N1≥1, the respective radio transponder being configured to transmit a radio signal having a certain frequency to a receiver, particularly adapted for restenosis identification.

For example, the implantable medical device is a stent, and the radio transponder is an RFID tag. In particular, stenosis refers to a narrowing or constriction of the diameter of a passage, e.g. a bodily passage or orifice. Further, restenosis may refer to the reoccurrence of stenosis in the passage, e.g. a blood vessel or heart valve after it has been treated (as by balloon angioplasty, stenting, or valvuloplasty).

By use of the N1 radio transponders, it is advantageously possible to monitor the health of the implantable medical device, in particular without the use of any radiation. Certain particles, like plague, inside and/or outside the implantable medical device may change characteristics of the radio signal transmitted by the radio transponder. Any change of the characteristics of the transmitted radio signal may be detectable by the receiver. Thus, such a change of the characteristics may indicate a change of the health of the implantable medical device.

To monitor the health of the implantable medical device, information describing the particles, e.g. plague formation, can be measured using said reader. In embodiments, this reader can be a hand-held device or part of an angiography system. The reader is adapted to measure the implantable medical device's health, e.g. plague formation, by detecting interference from the plague before and during follow-up check-ups. For example, the percentage of signal attenuation from a radio transponder placed on the implantable medical device can predict restenosis formation.

Using the present implantable medical device provides the advantage that no check angiogram is required during follow-ups for the patient. Furthermore, there is no need for contrast material injection into the veins for the identification of a blockage. Moreover, to verify the status of the implantable medical device, no additional X-ray or fluoroscopy is needed. For example, the present implantable medical device may be used in any stenting procedures, including coronary/neurovascular and peripheral stenting procedures.

According to an embodiment, the structure comprises a number of structural elements defining the cylindrical shape and the inner lumen. In particular, the structure comprises a pattern with a number of interconnecting struts defining the cylindrical shape and the inner lumen.

According to a further embodiment, the implantable medical device is an endoprosthesis. The endoprosthesis is a medical device that may be implanted inside the body of a patient to replace a damaged or missing part of a joint, bone, or other tissue. It may be used to restore function, relieve pain, or improve the quality of life for patients with conditions like arthritis, fractures, or joint degeneration, for example. The endoprosthesis can be made from materials like metal, plastic, or ceramic, and its design depends on the specific body part they are replacing.

According to a further embodiment, the implantable medical device is a stent.

A stent is an example of such an endoprosthesis. A stent is a cylindrically shaped device, which functions to hold open and sometimes expand a segment of a vessel, e.g. a blood vessel, or other anatomical lumen such as urinary tracts and bile ducts. A stent may be used in the treatment of atherosclerotic stenosis in blood vessels, for example. A stent, like a coronary stent for example, may have a diameter in the range from 2.5 mm to 4.5 mm and a length in the range of 12 mm to 48 mm. Such a stent with these dimensions may include multiple radio transponders.

According to a further embodiment, the implantable medical device comprises a plurality N1 of radio transponders, with N1≥2. Multiple radio transponders may be adapted to capture the data in a plurality of different directions (or angles) relative to the implantable medical device for calculating accurate and in-depth data on the implantable medical device, exemplary including stent restenosis, type of plague formation, percentage of plague and/or plague formation.

Using different angles may help determining the frequency range of each antenna, considering the data rate, read range, interference range, and/or attenuation at different intervals. In particular, the use of different angles may provide information if there is an excel blockage in between the implantable medical device after the procedure and post-checks.

According to a further embodiment, the N1 radio transponders are configured to transmit radio signals with N2 different frequencies, with 2≤N2≤N1. Using multiple radio transponders with multiple frequencies provides an improvement in calculating accurate and in-depth data on the implantable medical device. For example, if the implantable medical device includes five radio transponders, the five different frequencies may be 125 kHz, 128 kHz, 130 kHz, 12.6 MHz, and 13.53 MHz.

According to a further embodiment, the N1 radio transponders are configured to transmit radio signals with N1 different frequencies, wherein each of the N1 different frequencies is bijectively allocated to one of the N1 radio transponders. In other words, each of the plurality of radio transponders has its own frequency. As the allocation of radio transponders to frequencies is bijective, each one of the plurality of radio transponders corresponds to exactly one frequency, and vice versa.

According to a further embodiment, each of the N2 frequencies is selected from a frequency band between 100 kHz and 100 MHz. In embodiments, different frequencies or frequency bands are possible according to certain applications.

According to a further embodiment, the N1 radio transponders are configured to transmit radio signals with a common frequency. In some applications, it may be beneficial to use a plurality of radio transponders all using a common frequency, i.e. the same frequency, like 125 kHz.

According to a further embodiment, the radio transponder is an RFID tag. In particular, the RFID tag includes a substrate, an antenna, and a chip (or microchip). In particular, the RFID tag consists of the microchip, the substrate and the antenna being placed on said substrate. For example, the microchip of the RFID tag has a diameter of 1.4 mm and a length of 8.5 mm. For example, a coronary stent ranging from 2.5 mm to 4.5 mm in diameter, 12 mm to 48 mm length can accommodate multiple RFID tags. For example, the RFID chip antenna and the substrate may be built into the stent. In particular, the antenna and the substrate may be placed as part of the stent mesh and the chip of the RFID tag may be fixed in any side of the stent. For example, the RFID chip may have an area of 0.15×0.15 mm and a thickness of 7.5 μm. Such a chip is disclosed in reference [1], for example.

For the case that the radio transponders are embodied as RFID tags, the reader is embodied as an RFID reader. In particular, the maximum range of an RFID reader is 1.5 m without distraction. But once the present stent including at least one RFID tag is implanted, the reading capacity may vary and can be read effectively with an RFID reader placed close to the patient's body. It may be noted that the lifetime of a passive RFID tag can easily last over 20 years.

In particular, an RFID tag can store information, for example from 64 bits to 1 kB, in particular as the need to store customer data. With encryption, this data may be protected for medical needs so that the RFID tag information may not be exposed to any readers in the vicinity of the implantable medical device. As mentioned above, the RFID tags may use multiple frequencies, and further, the multiple RFID tags may be read from RFID readers arranged in different angles relative to the implantable medical device. Using these means, it is possible to provide accurate and in-depth data on stent restenosis, to accurately calculate a type of plague information (soft or hard plague), the percentage of plague, and/or extra plague in and around the stent area.

According to a further embodiment, the implantable medical device is embodied as a stent and the structure is embodied as a stent mesh. In particular, the substrate and the antenna of each of the N1 RFID tags are placed as part of the stent mesh or are placed in the inner lumen of the stent mesh.

According to a further embodiment, the respective RFID tag is embodied as a passive RFID tag. Advantageously, a passive RFID tag does not emit any radio frequency by default. A passive RFID tag installed inside a stent receives the radio frequency from the RFID readers and emit it. Particularly low frequencies (LF; low frequency) in a range between 128 kHz to 138 kHz may be used (see reference [2]). Particularly, the RFID tag antennas may be covered with polytetrafluoroethylene, which prevents restenosis. These RFID tags can be made liquid-proof with IPX8 rating to avoid tag failure post implant (see references [3] and [4]).

Any embodiment of the first aspect may be combined with any embodiment of the first aspect to obtain another embodiment of the first aspect.

According to a second aspect, a system, particularly adapted for restenosis identification, is suggested. The system comprises an implantable medical device according to the first aspect or to one of the embodiments of the first aspect, and a number N3 of readers, with N3≥1. Each of said N3 readers is configured to receive radio signals transmitted by the N1 radio transponders of the implantable medical device.

According to an embodiment, the system comprises a plurality N3 of readers, with N3≥2. In particular, the N3 readers are located in different angles relative to the implantable medical device. As mentioned above, with this arrangement of positioning a plurality of readers in different angles relative to the implantable medical device, it is possible to accurately calculate in-depth data on stent restenosis, type of plague formation, percentage of plague and/or extra plague formation.

According to a further embodiment, each of said N3 readers is configured to determine signal data based on the received radio signals transmitted by the implantable medical device. In particular, the signal data includes a signal strength, a signal attenuation, and/or a signal interference. By use of the detected signal strength, the detected signal attenuation, and/or the detected signal interior interference, one can derive a stent restenosis, type of plague formation, percentage of plague and/or extra plague formation.

According to a further embodiment, the system further includes a calculation unit which is configured to determine a restenosis information based on the signal data determined by the N3 readers. In particular, the restenosis information includes a first indicator indicating a percentage of plaque allocated to the implantable medical device, a second indicator indicating a type of the plaque allocated to the implantable medical device, and/or a third indicator indicating a plaque formation of the plaque.

The calculation unit may be implemented in hardware and/or in software. If said calculation unit is implemented in hardware, it may be embodied as a device, e.g. as a computer or as a processor or as a part of a system, e.g. a computer system. If said calculation unit is implemented in software, it may be embodied as a computer program product, as a function, as a routine, as a program code or as an executable object.

According to a further embodiment, at least one of the N3 readers is embodied as a separate hand-held device. For example, the hand-held device is an RFID reader.

According to a further embodiment, at least one of the N3 readers is embodied as part of an angiography system. In particular, the angiography system is an angiography solution for interventional radiology. For example, the angiography system is an ARTIS angiosystem.

The embodiments and features described with reference to the implantable medical device apply mutatis mutandis to the system.

Further possible implementations or alternative embodiments also encompass combinations—that are not explicitly mentioned herein—of features described above or below with regard to the embodiments. The person skilled in the art may also add individual or isolated aspects and features to the most basic form of the invention.

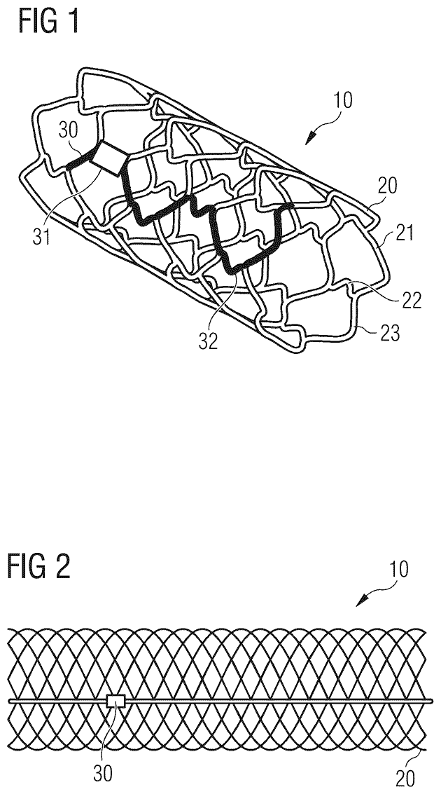

In FIG. 1, a schematic illustration of a first embodiment of an implantable medical device 10 is depicted. Particularly, the implantable medical device 10 is an endoprosthesis, for example a stent. In this regard, FIG. 2 shows a further view of the first embodiment of the implantable medical device 10 according to FIG. 1.

The implantable medical device of FIGS. 1 and 2 comprises a structure 20 having a cylindrical shape and an inner lumen. In particular, the structure 20 has a number of structural elements defining the cylindrical shape and the inner lumen. With respect to FIG. 1, only three of said structural elements 21-23 are comprised with reference signs to increase the readability of FIG. 1. As FIG. 1 further shows, the structure 20 may be embodied as a pattern having a number of interconnecting struts 21-23 defining the cylindrical shape and the inner lumen. For the case that the implantable medical device is embodied as a stent, as shown in FIGS. 1 and 2, the structure may be embodied as a stent mesh.

Furthermore, with reference to FIGS. 1 and 2, the implantable medical device 10 includes a number N of radio transponders 30, with N1≥1. Without loss of generality, the embodiment of FIGS. 1 and 2 has one radio transponder 30, with N1=1. The radio transponder 30 may be embodied as an RFID tag. The RFID tag 30 includes a substrate, an antenna 32 which is located on said substrate and a chip 31 (which may be also referred to as microchip or RFID chip).

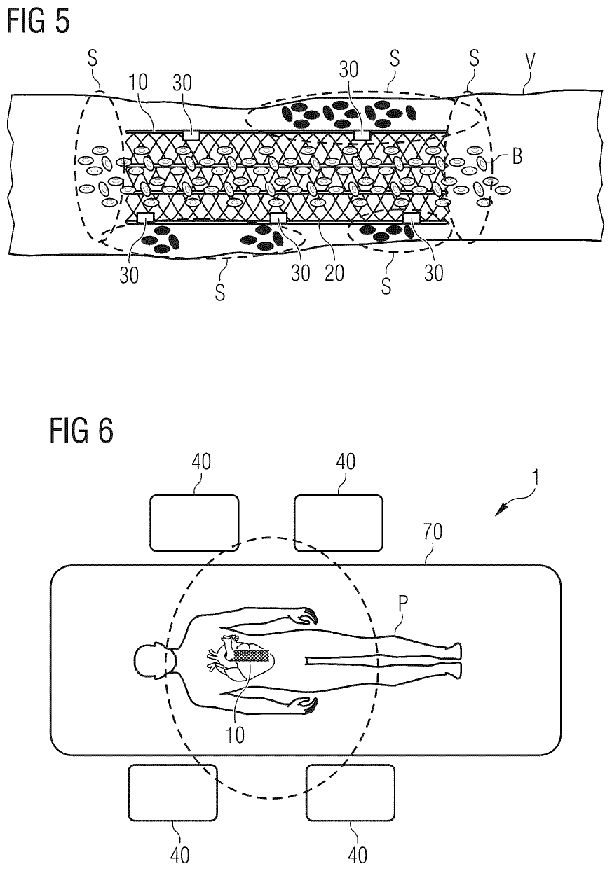

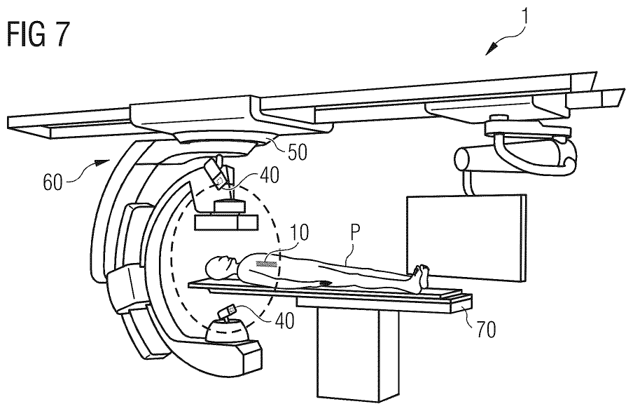

The radio transponder 30, for example embodied as RFID tag, is configured to transmit a radio signal having a certain frequence to a receiver 40 (see FIGS. 6 and 7). The transmitted radio signal is particularly adapted for restenosis identification, in particular in a case where the implantable medical device 10 is implanted in a blood vessel of a patient P (see FIG. 7).

For example, the substrate and the antenna 32 of the RFID tag 30 are placed as part of the stent mesh 20 of the stent 10. In alternative embodiments, the substrate and the antenna 32 of the RFID tag 30 are placed in the inner lumen of the stent mesh 20.

By use of the N1 radio transponders 30, it is advantageously possible to monitor the health of the implantable medical device 10, in particular without the use of any radiation. Certain particles, like plague, inside and/or outside the implantable medical device 10 may change characteristics of the radio signal transmitted by the radio transponder 30. Any change of the characteristics of the transmitted radio signal may be detectable by the receiver 40. Thus, such a change of the characteristics may indicate a change of the health of the implantable medical device 10.

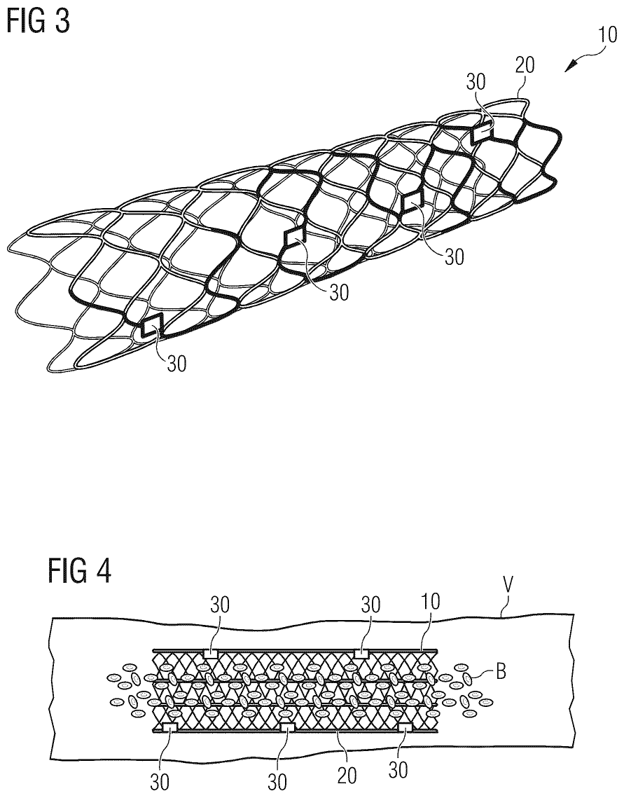

FIG. 3 shows a schematic illustration of a second embodiment of an implantable medical device 10 having a plurality of radio transponders 30. Each of the radio transponders 30 of FIG. 3 may be embodied as an RFID tag. The implantable medical device 10 of FIG. 3 is embodied as a stent. Without loss of generality, the stent 10 of FIG. 3 includes four radio transponders 30 (with N1=4), particularly embodied as a respective RFID tag 30. To improve readability, the single parts of the respective RFID tag, i.e. the RFID chip 31 and the antenna 32 with the underlying substrate, are not provided with dedicated reference signs in FIG. 3.

In particular, the N1 RFID tags 30 (with N1=4) of FIG. 3 are configured to transmit radio signals with N2 different frequencies, with 2≤N2≤N1. For example, each of the N2 frequences is selected from a frequency band between 100 kHz and 100 MHz.

In some applications, the N1 radio transponders 30 are configured to transmit radio signals with N1 different frequencies. In these cases, each of the N1 different frequencies is bijectively allocated to one of the N1 radio transponders 30. Multiple radio transponders 30 are configured to capture the data in a plurality of different directions (or angles) relative to the implantable medical device 10 to calculate accurate and in-depth data on the implantable medical device 10. The data may exemplarily include stent restenosis, type of plague formation, percentage of plague and/or plague formation.

In some alternative embodiments, the N1 radio transponders 30 are configured to transmit radio signals with a common frequency, e.g. 150 kHz.

With respect to FIG. 3, multiple RFID tags 30 with different frequencies or an equal frequency can be placed in or on the stent 10. With the help of an RFID reader 40 (see FIGS. 6 and 7) either hand-held or integrated into an angiography system 60, one can measure the strength of each RFID tag signal. Using RFID tags 30 with different frequencies may help identifying interference and attenuation due to plague or restenosis.

Furthermore, FIG. 4 shows a schematic illustration of a third embodiment of an implantable medical device 10 having a plurality of radio transponders 30 being implanted in a vessel V. Without loss of generality, the implantable medical device 10 of FIG. 4 is a stent, the radio transponders 30 are embodied as RFID tags, and the number of RFID tags 30 in FIG. 4 is five (N1=5).

If an RFID reader 40 (not shown in FIG. 4) is placed anywhere in the vicinity of the stent 10 of FIG. 4, the RFID reader 40 has a respective different angle relative to each one of the five RFID tags 30. Because of these different angles, a multi-direction detection is possible which provides an improved restenosis identification.

In this regard, FIG. 5 shows a further view of the third embodiment of FIG. 4 illustrating five different exemplary areas S of restenosis outside the stent area of the stent 10.

Using the multiple RFID tags 30 in the stent 10 and, therefore, the different angles to an RFID reader 40 provides that multi-direction detection and therefore the possibility to distinguish stenosis outside the stent area and stenosis inside the stent area.

Further, FIG. 6 shows a schematic illustration of a first embodiment of a system 1 adapted for restenosis identification. In this regard, FIG. 6 shows a patient P lying on a bed 70. In the vicinity of the bed 70, there are located four readers 40, for example embodied as RFID readers 40. Furthermore, FIG. 6 shows that an implantable medical device 10, e.g. a stent, is implanted in a vessel of the patient P laying on said bed 70. The four RFID readers 40 have different angles relative to said stent 10. In particular, said stent 10 has a plurality of radio transponders 30 as discussed with reference to FIG. 3 to 5. Each of said readers 40 is configured to receive radio signals transmitted by the radio transponders 30 of the stent 10.

The RFID readers 40 of FIG. 6 may be embodied as hand-held devices, for example, or may be integrated in an angiography system 60 which is discussed with reference to FIG. 7 in detail.

Thus, each of said readers 40 is configured to determine signal data including a signal strength, a signal attenuation and/or a signal interference based on the received radio signals transmitted by said stent 10. Based on the determined signal data, a restenosis information of said stent 10 may be determined. The restenosis information may include a first indicator indicating a percentage of plague allocated to the stent 10, a second indicator indicating a type of the plague allocated to the stent 10 and a third indicator indicating a plague formation of the plague.

Further, FIG. 7 shows a schematic illustration of a second embodiment of the system 1 adapted for restenosis identification. The system 1 of FIG. 7 includes an angiography system 60. Without loss of generality, the angiography system 60 of FIG. 7 comprises two RFID readers 40 arranged at the C-arm of the angiography system 60. The system 1 of FIG. 7 further includes a bed 70 on which a patient P is lying. A stent 10 is implanted in a vessel of said patient P. Radio signals transmitted by the RFID tags 30 of the stent 10 may be read by the RFID readers 40 of said angiography system 60.

The angiography system 60 may further include a calculating unit 50 which is coupled with said RFID readers 40. The calculation unit 50 may be adapted to determine a restenosis information based on the signal data determined by the RFID readers 40. The RFID readers 40 provide the signal data based on the received radio signals transmitted by the stent 10 of the patient P. The restenosis information provided by said calculation 50 may include above-discussed first indicator, the second indicator and/or the third indicator.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

### Specific Structural and Functional Details Disclosed

herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

### LIST OF REFERENCE

1 system
10 implantable medical device, e.g. stent
20 structure, e.g. stent mesh
21 structural element, e.g. strut
22 structural element, e.g. strut
23 structural element, e.g. strut
30 radio transponder, e.g. RFID tag
31 chip, e.g. RFID chip
32 antenna with underlying substrate
40 receiver, e.g. RFID receiver
50 calculation unit
60 angiography system
70 bed
B blood
P patient
S stenosis
V vessel

### REFERENCES

[1]

World's smallest and thinnest 0.15×0.15 mm, 7.5 μm thick RFID IC chip, Enhanced productivity enabled by ¼ surface area, ⅛th thickness, Feb. 6, 2006 (https://www.hitachi.com/New/cnews/060206.html#:~:text=HITACHI%20GLOBAL%20%3A%20News%20Release%20%3A%20World's, 7.5%C2%B5m%20thick%20RFID%20IC%20chip
[2]

https://www.arpansa.gov.au/understanding-radiation/what-is-radiation/non-ionising-radiation/radiofrequency-radiation [3]
[3]

https://www.encstore.com/blog/5820-understanding-ip-ratings-in-rfid-and-barcoding-devices
[4]

https://www.intechopen.com/chapters/45030

1. An implantable medical device, comprising:
a structure having a cylindrical shape and an inner lumen; and
a number of radio transponders, at least one radio transponder of the number of radio transponders being configured to transmit a radio signal having a certain frequency to a receiver.

2. The implantable medical device of claim 1, wherein the structure comprises a number of structural elements defining the cylindrical shape and the inner lumen.

3. The implantable medical device of claim 1, wherein the implantable medical device is an endoprosthesis or a stent.

4. The implantable medical device of claim 1, wherein the implantable medical device comprises a plurality of radio transponders.

5. The implantable medical device of claim 4, wherein a first number of radio transponders are configured to transmit radio signals with a second number of different frequencies, the second number being less than or equal to the first number.

6. The implantable medical device of claim 5, wherein the first number of radio transponders are configured to transmit radio signals with a same number of different frequencies, wherein each of the different frequencies is bijectively allocated to one of the first number of radio transponders.

7. The implantable medical device of claim 5, wherein each of the second number of frequencies is selected from a frequency band between 100 kHz and 100 MHz.

8. The implantable medical device of claim 4, wherein the number of radio transponders are configured to transmit radio signals with a common frequency.

9. The implantable medical device of claim 1, wherein each of the number of the radio transponders is a radio frequency identification (RFID) tag, the RFID tag including a substrate, an antenna, and a chip.

10. The implantable medical device of claim 9, wherein
the implantable medical device is a stent and the structure is a stent mesh, and
the substrate and the antenna of each of the number of RFID tags is placed as part of the stent mesh or in the inner lumen of the stent mesh.

11. The implantable medical device of claim 9, wherein each of the number of RFID tags is a passive RFID tag.

12. A system, comprising:
the implantable medical device of claim 1; and
a second number of readers, each of the readers being configured to receive radio signals transmitted by the number of radio transponders of the implantable medical device.

13. The system of claim 12, wherein the second number is greater than or equal to two, and the second number of readers are located in different angles relative to the implantable medical device.

14. The system of claim 13, wherein each of the plurality of readers is configured to determine at least one of signal data including a signal strength, a signal attenuation or a signal interference based on the received radio signals transmitted by the implantable medical device.

15. The system according to claim 14, further comprising:
a calculation unit configured to determine a restenosis information based on the signal data determined by the plurality of readers, the restenosis information including at least one of a first indicator indicating a percentage of plaque allocated to the implantable medical device, a second indicator indicating a type of the plaque allocated to the implantable medical device, or a third indicator indicating a plaque formation of the plaque.

16. The system of claim 12, wherein one of the second number of readers is a separate hand-held device or a part of an angiography system.

17. The implantable medical device of claim 1, wherein the at least one radio transponder of the number of radio transponders is configured to transmit the radio signal to the receiver to identify restenosis.

18. The implantable medical device of claim 2, wherein the structure includes a pattern with a number of interconnecting struts defining the cylindrical shape and the inner lumen.

19. The implantable medical device of claim 2, wherein the implantable medical device is an endoprosthesis or a stent.

20. The implantable medical device of claim 19, wherein the implantable medical device comprises a plurality of radio transponders.
